(19) United States
(12) Patent Application Publication
(10) Pub. No.: US 2006/0213775 A1
(43) Pub. Date: Sep. 28, 2006

(54) Solvent for preparation of analytical sample

(75) Inventors: Tetsuo Ohashi, Kyoto-shi (JP); Shin Nakamura, Kyoto-shi (JP)

Correspondence Address: KANESAKA BERNER AND PARTNERS LLP, SUITE 300, 1700 DIAGONAL RD, ALEXANDRIA, VA, 22314-2848, US

(73) Assignee: SHIMADZU CORPORATION, Kyoto-shi (JP)

(21) Appl. No.: 11/386,808

(22) Filed: Mar. 23, 2006

(30) Foreign Application Priority Data
Mar. 24, 2005 (JP) 2005-086638

(51) Int. Cl. G01N 27/00; C07K 1/26

(52) U.S. Cl. 204/601; 204/451

(57) ABSTRACT

A solvent for use in the preparation of an analytical sample includes at least one component selected from the group consisting of polyvalent alcohols, sugars, and hydrophilic polymers, and has a boiling point at one atmosphere of from 101° C. to 300° C. The solvent facilitates the preparation of samples for use in analyzing extremely small quantities in analytical devices having capillary channels. By virtue of its boiling point, the solvent can prevent the concentration of analytical samples without requiring special devices or tools, even when a minute quantity of sample solution is left for a long period of time or is placed in a high-temperature environment.

### BACKGROUND OF THE INVENTION AND RELATED ART STATEMENT

The present invention relates to a solvent for the preparation of samples, and is used when analyzing analytical samples in high-throughput type automatic analytical devices in fields such as medicine, biochemistry, food products, and the chemical industry.

In particular, the invention relates to a solvent for preparation of samples for use when analyzing extremely small quantities of samples such as proteins and amino acids in analytical devices having capillary channels in the fields of medicine and biochemistry.

In analysis with samples in a solution state, it is common to use water or an aqueous solution containing an inorganic or organic substance as a solvent for preparation of samples in order to dissolve the sample component. The component contained in the solvent for preparation of samples is added with the purpose of maintaining a state in which the structure or activity of the sample component that is the object of analysis is suitable for that analysis.

Meanwhile, recently, mechanical automation of the major analytical methods is advancing. In particular, analytical devices that use capillary tubes having an inner diameter of about 100 μm, and analytical devices called μ-TAS (micro total analysis system) or lab-on-chip, which use plate-shaped members (referred to below as capillary plates) having capillary channels having an inner diameter of about 100 μm formed inside, are being used as high-throughput type automatic analytical devices.

For example, although the base sequence decision method is the representative analytical method in the analysis of amino acids, it has come to be automated by devices referred to as sequencers, which have the capillary tubes or capillary plates as constituent elements, and furthermore, it has come to be processed in large volumes by making high throughput (see Japanese Unexamined PCT Patent Publication No. 2002-525574).

In the high-throughput type automatic analytical device, the inside of a fixed space of the device including the capillary tube or capillary plate may be kept constantly at a high temperature (in an electrophoresis device, a temperature of 50° C. or higher). In this case, in order to handle minute quantities of sample solution, transpiration of the water from the sample solution exposed to the gas phase occurs in a short time. Therefore, it is necessary to reduce the change of concentration, or the like, by assuring a fixed or higher quantity of sample solution. That advantage, however, is lost in these analytical devices characterized by minute quantity analysis.

Furthermore, in a high-throughput type automatic analytical device, the sample is usually a multi-specimen, and a micro titer plate having 96, 386 or 1536 wells is set inside the device. As a result, depending on the sample, it may be made to wait in the machine for a long period of time from the point of being set inside the machine until the analysis of all the other samples is finished. During that time, because transpiration of the water from the sample solution occurs and the sample solution is concentrated, not only is the analytical precision lowered by the change of concentration of the sample component, but there also is a risk of being connected to damage to the device.

In addition, analytical devices having special structures and tools are also used in order to prevent concentration of the sample solution in the sample container (see Japanese Unexamined Patent Publication No. 2003-166975).

The present invention was created in order to solve the above-described problems. An object of the invention is to provide a solvent for preparation of analytical samples supplied to an analytical device having micro capillary channels that makes it possible to prevent concentration of the analytical samples without requiring special devices or tools, even when a minute quantity of sample solution received in an open system container is left for a long period of time or is placed in a high-temperature environment.

Further objects and advantages of the invention will be apparent from the following description of the invention.

### SUMMARY OF THE INVENTION

According to one embodiment, the invention is a solvent for preparation of analytical samples supplied to an analytical device having capillary channels, and contains at least one component selected from a group consisting of polyvalent alcohols, sugars, and other hydrophilic polymers.

According to one aspect of the invention, the solvent has a boiling point at one atmosphere that is within the range of 101° C. to 300° C.

According to the invention, because at least one component selected from a group consisting of polyvalent alcohols, sugars, and other hydrophilic polymers is contained in the solvent for preparation of analytical samples, it is possible to raise the boiling point of the sample solution, and it is possible to prevent drying of the sample solution.

Also, according to another aspect of the invention, the specific gravity of the sample solution is increased. The container receiving the sample solution is stratified with water or a liquid having a specific gravity near that of water on the upper layer, and the sample solution on the lower layer is not diffused in the upper-layer liquid, so it is possible to prevent exposure of the sample solution to the gas phase.

### BRIEF DESCRIPTION OF THE DRAWINGS

FIGS. 1(A) and 1(B) show one example of a capillary electrophoresis device, to which a solvent for preparation of samples of the present invention is applied.

FIG. 2 shows another example applied to a capillary electrophoresis device, to which the solvent for preparation of samples of the present invention is applied.

FIGS. 3(A)-3(C) show an example of an electrophoresis device using a capillary plate as an electrophoresis member, to which the solvent for preparation of samples of the present invention is applied.

FIGS. 4(A)-4(D) show another example of an electrophoresis device using a capillary plate as an electrophoresis member, to which the solvent for preparation of samples of the present invention is applied, wherein FIG. 4(A) is a plan view of the capillary channels, FIG. 4(B) is an enlarged plan view of the sample reservoir (small-capacity reservoir) part on the cathode end, FIG. 4(C) is a perspective view of the cathode end, and FIG. 4(D) is a sectional view of the cathode end.

FIG. 5 is a sectional view of an end on the cathode side of another capillary plate.

FIG. 6 is a sectional view of an end on the cathode side of yet another capillary plate.

FIG. 7 is a sectional view showing operation of sample injection on the cathode side end in Working Example 1.

FIGS. 8(A)-8(C) show the results of electrophoretic analysis in Working Example 1.

FIGS. 9(A)-9(D) show the results of electrophoretic analysis in Working Example 2.

### DETAILED DESCRIPTION OF THE PREFERRED EMBODIMENTS

The solvent for preparation of analytical samples according to the present invention is used for preparation of analytical samples when performing analysis of sample components in the capillary channels of an analytical device.

Analytical devices having capillary channels include capillary electrophoresis devices using glass capillary columns having an inner diameter of about 100 μm as analysis channels (referred to below as a capillary electrophoresis device), as described in Japanese Unexamined PCT Patent Publication No. 2002-525574 and the specification of Japanese Unexamined Patent Publication No. H10-206384, which enable separation and analysis of extremely small quantities of components, and electrophoresis devices using MEMS (Micro Electro Mechanical System) capillary plates as electrophoresis members, as well as nano liquid chromatographs that use glass capillary columns or capillary plates, and devices for analysis of biological and pharmacological properties, environmental properties, and physical and chemical properties of compounds or substances contained in compounds.

The inner diameter of such capillary channels is 10 nm-1000 μm, and preferably 50 μm-100 μm.

Examples of analytical samples for analysis in those capillary channels include bio-molecules such as proteins, deoxyribonucleic acid (DNA), ribonucleic acid (RNA), and sugars, and chemical agents, drugs, and the like.

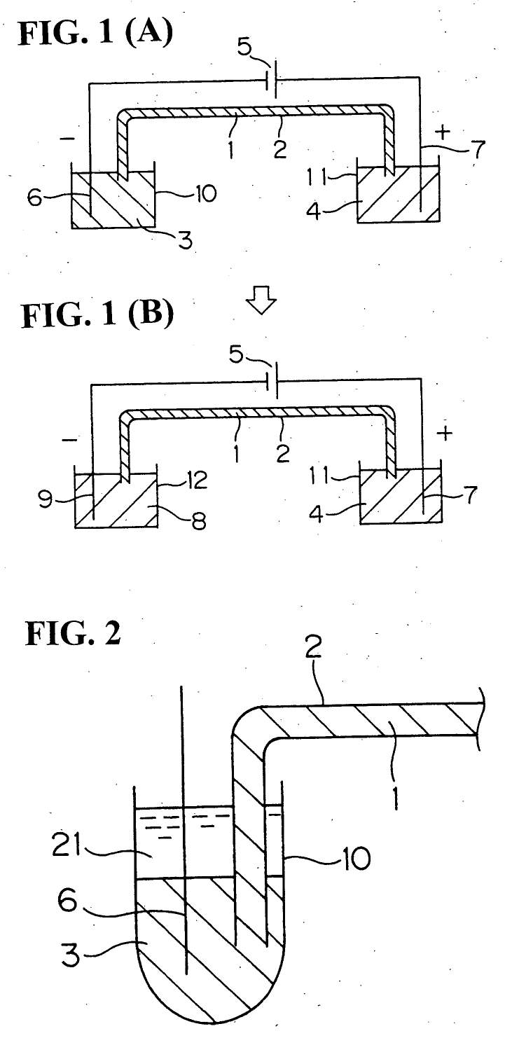

A first method of use of the solvent for preparation of samples of the present invention is explained conceptually with an example of a capillary electrophoresis device using one capillary column shown in FIGS. 1(A) and 1(B). First, in FIG. 1(A), one end of the capillary column 2 filled with phoresis medium 1 is immersed in a sample solution 3 being an analytical sample prepared using the solvent for preparation of samples of the present invention, and the other end of the capillary column 2 is immersed in a phoresis buffer 4.

After that, a prescribed voltage is applied between the sample solution 3 and the phoresis buffer solution 4 by a high-voltage electrophoresis power source 5 via electrodes 6 and 7, and the sample in the sample solution 3 is caused to move electrophoretically to one end of the capillary column 2, whereby injection of the sample into the capillary column 2 is performed.

After the sample is injected into one end of the capillary column 2, the application of voltage is stopped for a time, and the end on the sample injection side of the capillary column 2 is moved to a phoresis buffer 8 shown in FIG. 1(B), and is immersed.

After that, electrophoresis of the sample components is performed by applying high voltage between the phoresis buffers 4 and 8, and detection of the sample is performed by projecting measurement light or excited light and measuring the degree of light absorption or fluorescence using an optical measurement part not illustrated provided on the other end side of the capillary column.

FIG. 2 shows a second method of use of the solvent for preparation of samples of the present invention in a capillary electrophoresis device. FIG. 2 is an enlarged drawing of the end on the sample injection side of the capillary column 2 in FIGS. 1(A) and 1(B). In a well 10 receiving the sample solution, it is stratified with the sample solution 3 having prepared the sample with the solvent for preparation of samples of the present invention on the lower layer, and water or solution 21 having a specific gravity near that of water on the upper layer. At the time of sample injection into the capillary column 2, the front end on the sample injection side of the capillary column 2 is immersed into the sample solution 3 on the lower level inside the well 10. After that, voltage for sample injection is applied between the sample solution 3 and the phoresis buffer 4 by a high-voltage electrophoresis power source via electrodes 6 and 7, and injection of the sample into the capillary column is performed. The subsequent electrophoresis is performed in the same manner as in FIG. 1.

In the description above, injection of the sample into the capillary column was explained using an electrophoresis method, but the sample injection method is not limited to this method. In all cases in which a minute quantity of sample solution is received inside an unsealed container by a suction method, a pressurization method, and the like, it is possible to prevent drying of the sample solution by using the solvent for preparation of samples according to the present invention.

The inner diameter of the capillary tube is 10 nm-1000 μm, and preferably 50-100 μm. The well receiving the sample solution is preferably set so that the receivable solution becomes several 10 nL-several μL.

Also, in both cases of FIGS. 1(A) and 1(B), and FIG. 2, multiple samples can be received at one time by using a micro type plate having 96 or 386 wells for the well 10 receiving the sample solution 3. Furthermore, a plurality of those micro type plates may be stored inside the device, and in that case, the sample solution received in the sample container, depending on the sample, requires being held for a long time until it is injected into the capillary column. During this time, there is a possibility that the solvent may transpire from the sample solution and concentration of the sample may occur. But, by preparing the sample using the solvent for preparation of samples of the present invention, drying of the sample solution is prevented and improvement of analytical precision is possible. Also, it becomes possible to allow a large number of sample solutions to wait in the analytical device, and the number of times of packing of samples into the analytical device by hand is reduced, whereby improvement of operating efficiency is achieved.

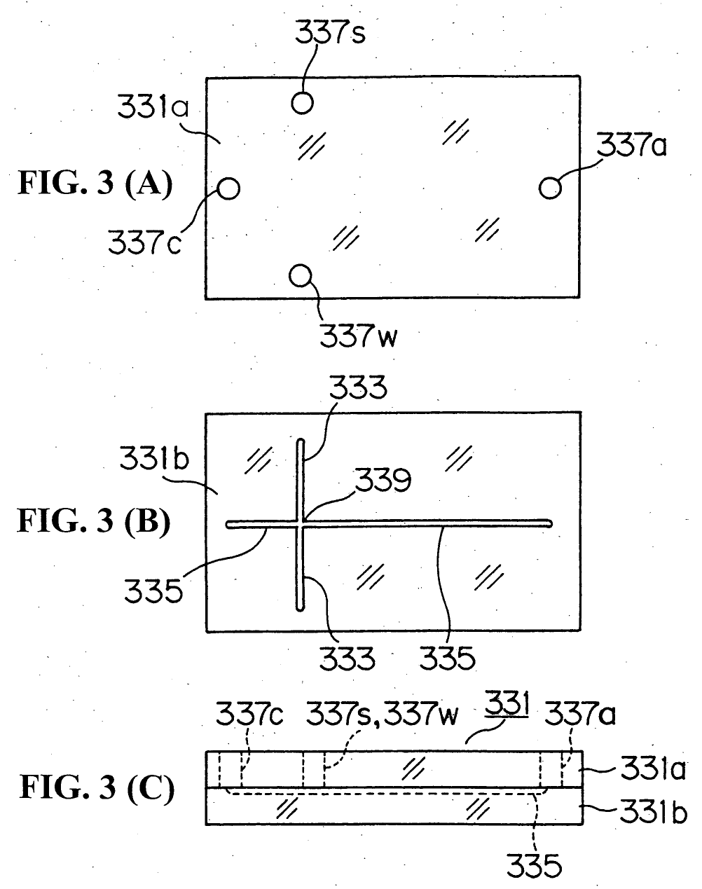

Next, a method of use in a device having a MEMS capillary plate as an electrophoresis member, as another analytical device having capillary channels that uses the solvent for preparation of samples according to the present invention, is explained while referring to FIGS. 3(A)-3(C).

The phoresis member consists of a pair of plate-shaped members 331a and 331b. FIG. 3(A) is a top view of the plate-shaped member 331a, FIG. 3(B) is a top view of the plate-shaped member 331b, and FIG. 3(C) is a sectional view of the phoresis member. On the top surface of one member 331b, mutually intersecting capillary phoresis channels 333 and 335 are formed by photolithography technology used in semiconductor fabrication processes or by micromachining technology.

On the other member 331a, through-holes are provided in positions corresponding to the ends of the channels 333 and 335 as anode reservoir 337a, cathode reservoir 337c, sample reservoir 337s, and sample waste reservoir 337w. In advance of sample injection, for example, the phoresis medium is filled into the capillary channels 333 and 335 from any reservoir by pressurization using a syringe. Next, the phoresis medium filled inside the reservoir is removed, the sample solution is injected into the sample reservoir 337s corresponding to one end of the shorter capillary channel 333 (sample injection channel), and phoresis buffer solution is injected into the other reservoirs 337w, 337c, and 337a. After that, a prescribed voltage is applied to each reservoir 337s, 337w, 337a, and 337c, and the sample is led to the intersecting part 339 of the two capillary channels. After the intersecting part 339 is filled with the sample, the voltage applied to each reservoir is switched and the sample in the intersecting part 339 is injected into the longer capillary channel 335.

The width of the capillary channel is 10 nm-1000 μm, and preferably 50 μm-100 μm. The capacity of each reservoir is preferably set so that the receivable solution becomes several 10 nL-several μL.

The following describes a second method of use of the solvent for preparation of samples according to the present invention in an electrophoresis device using a capillary plate as electrophoresis member. After filling the phoresis medium into the capillary channel and cleaning the phoresis member filled into each reservoir, water is first put into the sample reservoir 337s. After that, sample solution, having prepared the sample with the solvent for preparation of samples of the present invention, is put into the bottom of the sample reservoir, and it is made to be stratified with the sample solution on the lower layer and the water on the upper layer.

Next, the solvent for preparation of samples according to the present invention is explained.

The solvent for preparation of analytical samples according to the present invention is constituted mainly by water, and the solvent contains at least one or more components selected from a group consisting of polyvalent alcohols, sugars, and other hydrophilic polymers.

Examples of the polyvalent alcohols, bivalent alcohols and trivalent alcohols include, for example, ethylene glycol, glycerol, pentaerythritol, propylene glycol, and mannitol, and the like.

Polyvalent alcohols mix easily with water, and they have a higher specific gravity than water. Also, because the boiling point of polyvalent alcohols is higher, the boiling point of ethylene glycol being 198° C. and the boiling point of glycerin being 290° C., and because the sublimation property is low, transpiration of the water is suppressed, and drying of the sample solution can be prevented.

Although from the past formamide has been added to solvents for preparation of samples for electrophoresis using micro capillary tubes or capillary plates, just as in the traditional slab gel electrophoresis, the reactivity is higher compared with polyvalent alcohols, and it is chemically unstable. Also, the effects on the body and the environment are high, with reports of liver dysfunction caused by inhalation of formamide vapor, and the like.

On the other hand, polyvalent alcohols have little effect on sample components of bio-molecules such as proteins and amino acids, and they have excellent characteristics also in terms of sample storability. In addition, they tend not to produce decomposition products, and they tend not to influence phoretic separation of analytical samples. Furthermore, the effect on the environment can be minimized because they have good biodegradability, and they also have low toxicity to the ecology.

In the case in which only polyvalent alcohol is added as an additive component, it is contained in the solvent for preparation of samples preferably at 5-80 (w/v) %, more preferably at 20-60 (w/v) %, and even more preferably at 35-55 (w/v) %.

Examples of sugars include monosaccharides, and oligosaccharides and polysaccharides having a plurality of these condensed, are included. Specifically, examples of monosaccharides include glucose, mannose, galactose, glucosamine, and derivatives thereof, and the like. Examples of oligosaccharides include lactose, sucrose, trephalose, and derivatives thereof, and the like. Examples of polysaccharides include dextran, cellulose, chitin, chitosan, and derivatives thereof, and the like.

Sugars, just as polyvalent alcohols, mix easily with water, and they can raise the boiling point of the solvent.

In the case in which only sugar is added as an additive component, it is contained in the solvent for preparation of samples preferably at 5-80 (w/v) %, more preferably at 20-60 (w/v) %, and even more preferably at 35-55 (w/v) %.

Examples of other hydrophilic polymers include polysilane, polyvinyl pyrrolidone, and the like.

The components added to the solvent for preparation of samples also can be made as plural components selected from a group consisting of polyvalent alcohols, sugars, and other hydrophilic polymers.

Also, the solvent for preparation of samples according to the present invention contains at least one or more components selected from a group consisting of polyvalent alcohols, sugars, and other hydrophilic sugars, and preferably the boiling point at one atmosphere is 101-300° C., and more preferably 120-250° C.

By making the boiling point at one atmosphere 101° C. -300° C., concentration of the sample solution can be prevented even in the case in which sample solution on a microliter order is put into the sample container and is left in a high-temperature environment, or when it is left for a long period of time.

Working examples are shown below and the present invention is specifically explained, but the present invention is not to be limited to the working examples described below.

### WORKING EXAMPLE 1

In the present working example, a DNA sample was prepared using the solvent for preparation of samples according to the present invention. Electrophoresis was performed by the Sanger method in an electrophoresis device having a capillary plate as a constituent element.

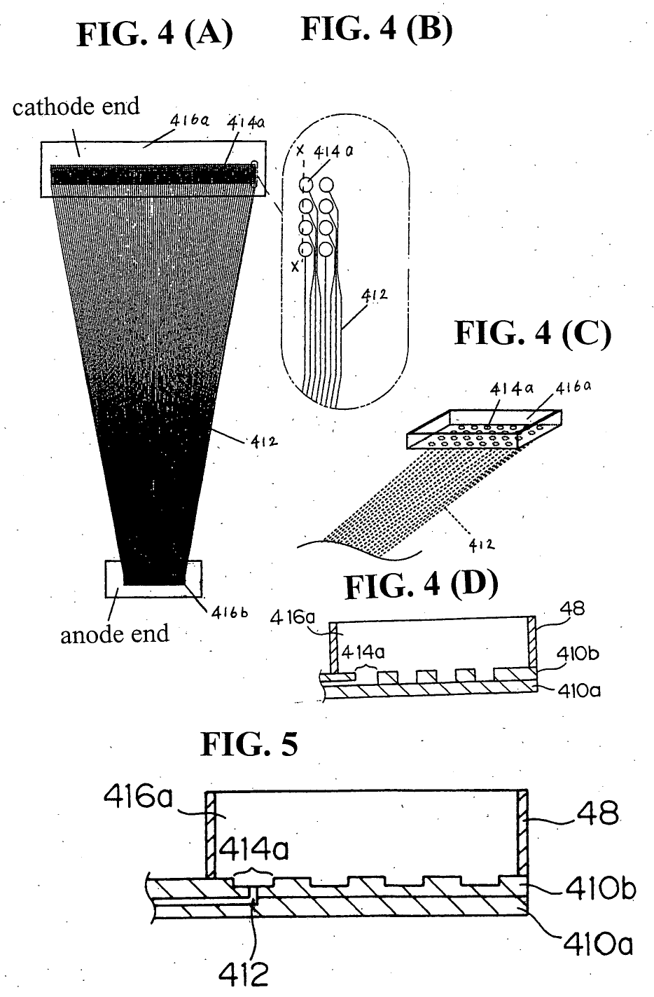

FIGS. 4(A)-4(D) show the electrophoresis member used in the present working example. FIG. 4(A) shows a plan view of the capillary channels in the electrophoresis member consisting of a capillary plate, FIG. 4(B) shows an enlarged plan view of the sample reservoir part on the cathode end, FIG. 4(C) shows a perspective view of the cathode end, and FIG. 4(D) shows a sectional view of the cathode end.

The electrophoresis member has a pair of plate members 410a and 410b bonded together. On one plate member 410a, multile, for example 384, separation channels 412 consisting of capillary channels are formed, and they are arranged so as not to intersect with each other.

One end of each separation channel 412 is connected to a respective sample reservoir (referred to below as a small-capacity reservoir) opened on the substrate surface. On the substrate surface, a large-capacity reservoir 416a having a size encompassing a plurality of small-capacity reservoirs 414a is formed being surrounded by a wall 48. The other end (anode end) of each separation channel 412 is opened so as to be connected to a common reservoir 416b formed on the substrate surface.

The width of the separation channel is 10 nm-1000 μm, preferably 50-130 μm, and the depth is 10 nm-1000 μm, preferably 20 μm-60 μm.

On the other plate member 410b, through-holes are formed in positions corresponding to the two ends of the separation channels 412. The through-holes on one end side are the small-capacity reservoirs 414a, and the size of the small-capacity reservoir 414a is a diameter of 10 μm-3 mm, preferably 50 μm-2 mm, and it is set to a size suitable for injecting several 10 nL-several μL of sample. Both plate members 410a and 410b are affixed together with the separation channels 412 on the inside so as to become a single member.

Formation of the separation channels 412 on the plate member 410a can be done by lithography and etching (wet etching or dry etching). Formation of the through-holes on the plate member 410b can be done by a method such as sand blasting or laser drilling.

The entire area of the small-capacity reservoirs 414a is covered by the large-capacity reservoir 416a, and as in FIG. 4(C) showing the perspective view, all the small-capacity reservoirs 414a are provided inside the large-capacity reservoir 416a, and they are connected with the reservoir 416a.

The reservoir 416b on the other end side also covers the area where the openings on the other end side of all the separation channels 412 are disposed, and the openings on the other end side of all the separation channels 412 are connected with the reservoir 416b.

As for the material of the plate members 410a and 410b constituting the substrate, quartz glass or borosilicate glass, resin, or the like, can be used, and a transparent material is selected in the case when the components separated by phoresis are detected optically. In the case of using a detecting means other than light, the material of the plate members 410a and 410b is not limited to one that is transparent.

The inner wall of the small-capacity reservoir 414a may be made hydrophilic, and the bottom surface of the large-capacity reservoir 416a or from the bottom surface to the inner wall surface may be made hydrophobic.

As for the surface treatments for such hydrophilic and hydrophobic properties, various methods can be mentioned. For example, in the case of using a glass plate as the plate member, the hydrophilic property can be given by treating with acid, and the hydrophobic property can be given by coating with resin, processing with fluorine resin or treating with silane coupling agent, or the like.

FIG. 5 shows a sectional view on the cathode side of another capillary plate. The small-capacity reservoir 414a is formed as a cavity on the top surface side of the plate member 410b, and it is connected at the bottom with the separation channel 412. Multiple small-capacity reservoirs 414a are covered by a large-capacity reservoir 416a, and they are formed on the bottom surface of the large-capacity reservoir 416a.

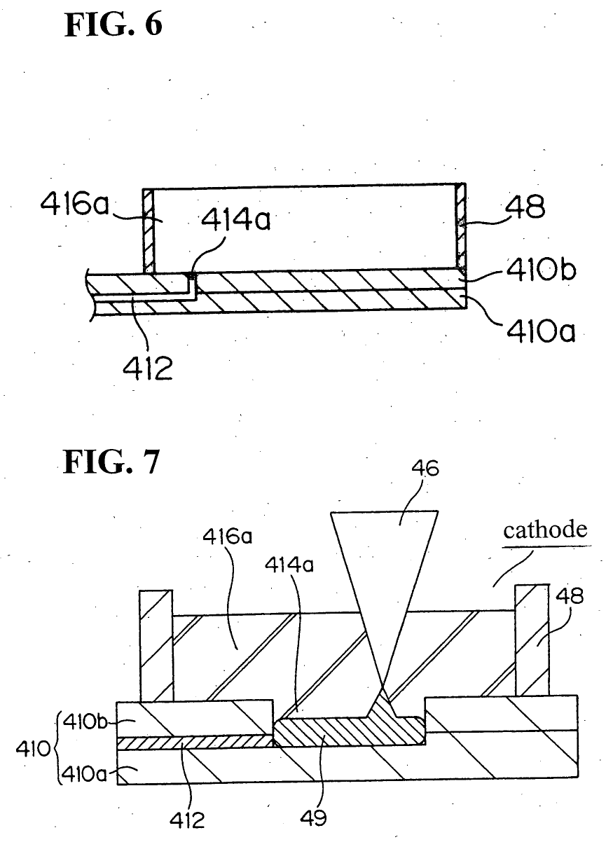

FIG. 6 shows a sectional view on the cathode side of yet another capillary plate. The small-capacity reservoir 414a is formed as an opening having a size about the same as that of the separation channel 412.

In either of these capillary plates shown in FIG. 5 or FIG. 6, surface treatment may be applied so that the small-capacity reservoir 414a and a narrow range of the periphery of the opening of the small-capacity reservoir 414a on the bottom surface of the large-capacity reservoir 416a become hydrophilic, and the outside of that becomes hydrophobic. By this, the injected sample solution comes to be held in the part applied with hydrophilic treatment, and that hydrophilic area becomes the small-capacity reservoir. The size of that hydrophilic area is set to a size suitable for the quantity of sample to be held, typically several 10 nL-several μL.

Next, the sample injection operation in the capillary plate shown in FIG. 4 is explained while referring to FIG. 7.

(1) The capillary plate is kept in a constant-temperature state of 50° C.

(2) The large-capacity reservoir 416a on the cathode side is filled with pure water, for example Milli-Q water, and phoresis medium is packed into all the separation channels 412 by pressurizing by syringe from the anode side.

(3) Because the phoresis medium flowing out from the separation channels 412 to the small-capacity reservoirs 414a diffuses in the pure water of the large-capacity reservoir 416a, the water and the phoresis medium inside the reservoirs 414a and 416a are drawn by a suction nozzle, and the insides of the reservoirs 414a and 416a are cleaned.

(4) After cleaning the insides of the reservoirs 414a and 416a, phoresis buffer solution is filled into the cathode-side reservoir 416a and the anode-side reservoir 416b, voltage is applied between the two reservoirs 414a and 416b to perform pre-separation, and ions of impurities in the gel are caused to move toward the anode electrode or the cathode electrode. The applied voltage is, for example, 125 V/cm, and the application time is 5 minutes.

(5) The phoresis buffer solution in the cathode-side reservoir 416a is drawn, and the inside of the reservoir 416a is cleaned, and then the inside of the reservoir 416a is filled with pure water, for example Milli-Q water.

(6) After that, sample solution 49 is dripped successively or in units of pluralities by pipetter 46 into each small-capacity reservoir 414a of the reservoir filled with pure water. Dripping of the sample solution is performed by lowering the front end of the pipetter 46 to near the small-capacity reservoir 414a, for example to a position at a distance of about 0.5 mm from above. Also at this time, the capillary plate is maintained in a constant-temperature state of 50° C.

(7) A cathode electrode is inserted into each small-capacity reservoir 414a, and phoresis voltage is applied between it and the anode electrode to perform sample injection into the channel 412. The applied voltage for sample injection is, for example, 50 V/cm, and the application time is 40 seconds.

(8) After drawing the pure water in the reservoir 416a as well as the remaining samples in the small-capacity reservoirs 414a and cleaning, the reservoirs 414a and 416b are filled with phoresis buffer.

(9) A cathode electrode is inserted into the reservoir 414a, and phoresis voltage is applied between it and the anode electrode to perform electrophoresis separation and signal detection of the sample. The applied voltage for phoresis separation is suitably 70-300 V/cm, for example, 125 V/cm.

The electrode may be provided in advance respectively in the reservoirs 416a and 416b, and it also may be inserted separately. Also, on the sample injection side, an electrode may be provided for each reservoir 414a, and it also may be inserted separately.

The measurement conditions are as follows.

The DNA sample was prepared using a BigDye Terminator Cycle Sequencing Ready Reaction Kit v3.1 reagent kit for cycle sequencing (manufactured by Applied Biosystems Corporation). The cast DNA was pUC18 plasmid DNA (manufactured by Toyobo Corporation), and (synthetic product entrusted to QIAGEN) was used for the primer. The reaction capacity was set to 2.5 μL. The other conditions followed the kit handling instructions, and standard product made by performing ethanol precipitation processing and then drying and hardening was obtained.

As solvents for preparation of samples for sequencer, the following three kinds of solvents were prepared.

(a) Aqueous solution containing 50% (w/v) ethylene glycol, and each component of 0.4 mM Tris-HCl (pH 8.0) and 0.04 mM EDTA.

(b) Aqueous solution containing 60% (w/v) formamide, and each component of 0.4 mM tris-HCl (pH 8.0) and 0.04 mM EDTA.

(c) Distilled water (solvent for preparation of sample for comparison).

The above dry standard product of DNA sample was dissolved using 5 μL of the aforementioned three kinds of solvents for preparation of samples in (a) to (c), and sample solution to supply to the sequencer was prepared. Then, 2.3 μL of each sample solution was injected by mechanically-controlled pipetter 46 into the small-capacity reservoirs 414a formed on the BioMEMS capillary plate of the sequencer shown in FIG. 4 as described above.

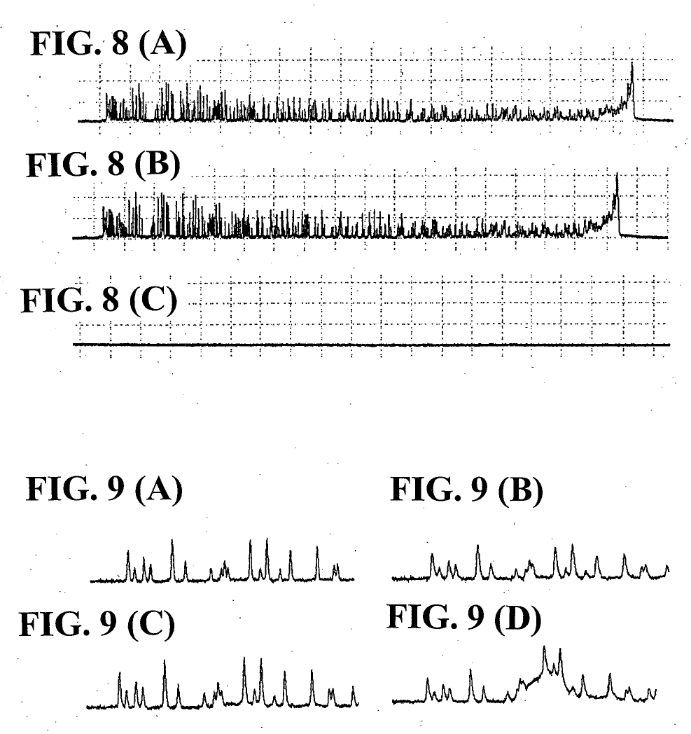

FIGS. 8(A)-8(C) show the phoresis results. Each sequencing data indicates the fluorescence signal for G of the four kinds of fluorescence signals of pUC18 plasmid DNA. The vertical axis in the illustrated graph indicates the fluorescence strength. Also, the horizontal axis indicates the detection time, and the left side of the graph is the starting point of analysis.

FIG. 8(A) is the electrophoresis data of the sample using the solvent (a) for preparation, FIG. 8(B) is the electrophoresis data of the sample using the solvent (b) for preparation, and FIG. 8(C) is the electrophoresis data using distilled water being solvent (c) for preparation. Although normal sequencing data was obtained in FIG. 8(A) and FIG. 8(B), no signals were observed in the sample solution prepared with water in FIG. 8(C). This is thought to be because at the time of ejection of the sample solution into the water from the pipetter tip, the specific gravity of the sample solution was nearly the same as water, and therefore it diffused into the liquid inside the reservoir 416a before the DNA sample moved to the phoresis medium. Although equivalent results were obtained for FIG. 8(A) and FIG. 8(B), it is because ethylene glycol and formamide each have a higher specific gravity than water, and the two have nearly the same specific gravity.

### WORKING EXAMPLE 2

In the second working example, a comparison regarding the resistance to drying of the solvent for preparation of samples of the present invention was performed.

Although the electrophoresis member used in the present working example is the same as in Working Example 1, the capillary plate was kept at room temperature until the start of phoresis. The operation of sample injection is the same as in steps (2) to (4) of Working Example 1, and from step (5) onward, it is as follows.

(5) After drawing the buffer solution in the cathode-side reservoir 416a and cleaning the inside of the reservoir 416a, 5 μL of sample solution 49 is dripped by pipetter 46 into the reservoirs 414a.

(6) After setting aside for 24 hours with the top surface of the reservoirs 414a in an open condition, a cathode electrode is inserted into each small-capacity reservoir 414a, and voltage is applied between it and the anode electrode to perform sample injection into the channel 412. The applied voltage for sample injection is, for example, 50 V/cm, and the application time is 40 seconds.

(7) After drawing the remaining samples in the small-capacity reservoirs 414a and cleaning, the insides of the reservoirs 414a and 416a are filled with buffer solution.

(8) A cathode electrode is inserted into the reservoir 416a, and phoresis voltage is applied between it and the anode electrode to perform electrophoresis separation and signal detection of the sample.

The DNA sample preparation conditions and the solvent for preparation of sample for sequencer were prepared under the same conditions as Working Example 1.

FIGS. 9(A)-9(D) show the phoresis results. Each sequencing data indicates the fluorescence signal for G of the four kinds of fluorescence signals of pUC18 plasmid DNA. The vertical axis in the illustrated graph indicates the fluorescence strength. Also, the horizontal axis indicates the detection time, and the right side of the graph is the signal on the long chain side.

FIG. 9(A) is the electrophoresis data of the sample prepared using the solvent (a) before setting aside at room temperature. FIG. 9(B) is the electrophoresis data of the sample prepared using the solvent (a) after setting aside for 24 hours at room temperature. FIG. 9(C) is the electrophoresis data of the sample prepared using the solvent (b) before setting aside at room temperature. FIG. 9(D) is the electrophoresis data of the sample prepared using the solvent (b) after setting aside for 24 hours at room temperature.

With the sample prepared with the solvent for preparation of samples containing ethylene glycol in (a), and the sample prepared with the solvent for preparation of samples containing formamide in (b), signals indicating that fluorescence-tagged reaction products were detected could be confirmed, but because the sample solution prepared with water being solvent (c) completely dried up inside the microplate well, it became incapable of measurement.

On the other hand, when comparing the results of the solvents for preparation of samples (a) and (b), a rise of the baseline thought to be a decomposition product of formamide was observed near 300 bases in the data of the solvent for preparation of samples (b) shown in FIG. 9(D). There is a possibility that this phenomenon may cause misunderstanding of the base sequence in decoding of the sequencing data performed by computer. In the case prepared using the solvent for preparation of samples (a), compared with the case prepared with the solvent for preparation of samples (b), it could be confirmed that it can withstand setting aside at room temperature for a longer period without influencing analysis.

Although ethylene glycol is one kind of polyvalent alcohol, it is thought that other polyvalent alcohols such as glycerol, pentaerythritol, propylene glycol, and mannitol, which have similar chemical properties, also have the same kind of property.

The disclosure of Japanese Patent Application No. 2005-086638 filed on Mar. 24, 2005, is incorporated herein.

While the invention has been explained with reference to the specific embodiments of the invention, the explanation is illustrative and the invention is limited only by the appended claims.

What is claimed is:

1. A solvent for preparing an analytical sample, comprising:
at least one component selected from the group consisting of polyvalent alcohols, sugars, and hydrophilic polymers.

2. A solvent according to claim 1, wherein the solvent has a boiling point at one atmosphere of from 101° C. to 300° C.

3. A solvent according to claim 1, wherein the at least one component is a polyvalent alcohol present in an amount of from 5-80 (w/v) %.

4. A solvent according to claim 1, wherein the polyvalent alcohol is selected from the group consisting of ethylene glycol, glycerol, pentaerythritol, propylene glycol, and mannitol.

5. A solvent according to claim 1, wherein the at least one component is a sugar present in an amount of from 5-80 (w/v) %.

6. A solvent according to claim 1, wherein the sugar is selected from the group consisting of monosaccharides, oligosaccharides, and polysaccharides.

7. A solvent according to claim 1, wherein the hydrophilic polymer is selected from the group consisting of polysilane and polyvinyl pyrrolidone.

8. A solvent according to claim 1, further comprising water.
